(19) United States
(12) Patent Application Publication
(10) Pub. No.: US 2019/0254928 A1
(43) Pub. Date: Aug. 22, 2019

(54) DRINKING STRAW FOR ADMINISTERING AN ACTIVE SUBSTANCE

(71) Applicant: DS-Technology GmbH, Winnenden (DE)

(72) Inventors: Matthias MOESSINGER, Allmersbach im Tal (DE); Daniel MUELLER, Allmersbach im Tal (DE); Jan PAETZOLD, Allmersbach im Tal (DE); Elke STERNBERGER-RUETZEL, Allmersbach im Tal (DE)

(73) Assignee: DS-Technology GmbH, Winnenden (DE)

(21) Appl. No.: 16/276,862

(22) Filed: Feb. 15, 2019

(30) Foreign Application Priority Data
Feb. 21, 2018 (EP) 18 000 176.0

(51) Int. Cl. A61J 7/00; A47G 21/18

(52) U.S. Cl. CPC A61J 7/0038; A61M 2202/064; A47G 21/183

(57) ABSTRACT

A drinking straw for administering an active substance has a wall with a first end and with a second end. One of the two ends of the wall can be closed releasably with a closure element. A membrane, on which the active substance lies, is present in the region of the other end of the wall.

### CROSS REFERENCE TO RELATED APPLICATIONS

Applicant claims priority under 35 U.S.C. § 119 of European Application No. 18 000 176.0 filed Feb. 21, 2018, the disclosure of which is incorporated by reference.

### BACKGROUND OF THE INVENTION

### 1. Field of the Invention

The present invention relates to a drinking straw for administering active substances, for example food supplements or medicaments. The active substance to be administered is already located in the drinking straw and is sucked with a liquid from the drinking straw into the mouth and then swallowed. This generally makes it easier to take the active substance, since the latter can be much more easily swallowed. By providing the active substance as an in situ suspension with a liquid, it is additionally possible for the unpleasant taste of the active substance to be masked by the liquid.

### 2. Description of the Related Art

Drinking straws for administering active substances are known from WO 98/51259 A1 or WO 2006/079648 A1, for example. The drinking straws described there have a wall with a first end and a second end, wherein the wall can have a reinforcement at the first and/or the second end. A controller is located in the interior of the drinking straw. This controller prevents the active substance, likewise located in the drinking straw, from falling out at the bottom of the drinking straw. The controller is originally located in the lower region of the drinking straw. During the process of drinking, the controller is sucked upwards and also remains at this new position after the drinking straw has been set down. From the position of the controller, it is thus easy to establish whether or not the active substance has already been completely consumed.

The controllers are generally configured as open-pore solid bodies and made of a gas-permeable and/or liquid-permeable material. Thus, the controllers can be configured, for example, in the form of a small sponge or in the form of a fibrous mesh.

The controllers have hitherto been developed for the administration of active substances in the form of pellets. The pellets used have a defined minimum size and a defined particle size distribution. By contrast, in the administration of active substances in the form of fine powders, it can happen that these powders trickle through the controller and, for example, escape into the packaging of the drinking straw even before the process of sucking has been started. Moreover, it is not generally possible to ensure that the fine powders are completely consumed, since some of the fine powder can remain attached to the controller. Particularly in the administration of potentially toxic medicaments, for example oncology drugs, this can place third parties in danger, since these third parties may thus accidentally come into contact with the active substance. Consequently, the administration of active substances in the form of fine powders with a particle size of under 210 micrometres is not possible with the described controllers.

### SUMMARY OF THE INVENTION

Proceeding from this known prior art, the object of the invention was to make available a drinking straw for administering an active substance, which drinking straw is such that active substances in the form of fine powders can also be safely administered.

The drinking straw according to the invention for administering an active substance is defined by the features of the main claim, i.e. Claim 1. Advantageous developments of the invention form the subject matter of further claims that are dependent on this main claim.

The drinking straw according to the invention for administering an active substance has a wall with a first end and with a second end, and a closure element with which one of the two ends of the wall can be releasably closed. According to the invention, a membrane is present in the region of the other end of the wall. The active substance can lie on this membrane, such that the active substance is enclosed in the interior of the wall between the membrane and the closure element. The use of a membrane instead of the controller makes it possible even for fine powders to be administered safely and in exact doses by means of the drinking straw according to the invention.

In a first embodiment, the membrane can be permeable to liquid. The membrane can therefore remain intact during the drinking process, since the liquid that is drunk in order to permit consumption of the active substance can pass through the membrane. In this way, it is also possible to ensure that no undesired solids are accidentally consumed too.

In a second embodiment, the membrane, during the sucking process, can form an opening through which liquid can pass through the membrane. In this case, the membrane can, for example, burst open or tear in a controlled manner, as a result of which the corresponding opening is able to form. To allow the formation of the opening to proceed in a controlled manner, the membrane can preferably have a predetermined breaking point, which can be configured in particular as a material thinning. Such a predetermined breaking point in the form of a material thinning can be provided, for example, through removal of material by means of a laser. The predetermined breaking point should be located in particular in the central region of the membrane and not at the edge regions of the latter. In this way, it is possible to prevent the membrane from detaching wholly or partially from the wall of the drinking straw.

Independently of the configuration of the membrane, the membrane in a first embodiment can be secured to the front face of the wall of the drinking straw. Such securing can be provided, for example, by a suitable adhesive or by sealing the membrane onto the front face.

In order to avoid subsequent manipulation of the membrane after the drinking straw has been filled with the active substance, the membrane in a preferred embodiment can also be arranged offset by a short a distance to the inside and can thus be secured to the inner wall of the drinking straw.

In an embodiment with a particularly simple design, the membrane can be secured to the inner wall of the drinking straw by means of a clamping element. Such a clamping element can in particular have a tubular fitting, the circular external diameter of which is slightly smaller than the circular internal diameter of the wall of the drinking straw. If such a tubular fitting is provided with a continuous slit in the longitudinal direction, the tubular fitting expands such that its external diameter increases. The tubular fitting can in this case be compressed by application of just a slight force and in this way inserted a distance into the interior of the wall. After the tubular fitting has been positioned, it is able to expand and can press tightly against the inner wall of the drinking straw. As a result, a membrane that has been placed over the tubular fitting can be securely fixed inside the drinking straw. In the simplest case, the membrane can lie loosely over the tubular fitting of the clamping element. As an alternative to this, the membrane can also be secured to the tubular fitting by means of an adhesive, for example. It would also be possible to seal the membrane onto the tubular fitting.

To prevent a situation in which, during storage or transport, the active substance trickles out of the drinking straw from the end of the wall opposite the membrane, the corresponding end of the wall is closed by means of a closure element. The closure element can be removed by the patient or a helper directly before the active substance is consumed, such that this end of the drinking straw can be taken into the patient's mouth. In the simplest case, the closure element can be a cap, which can be pushed with frictional engagement over the end of the wall. To prevent as effectively as possible a situation in which the fine powder trickles out, the closure element can preferably be configured as a foil. The foil can be secured releasably to the front face of the wall. Such securing can be provided, for example, by a suitable adhesive or by sealing the foil on. As an alternative to this, the closure element can be secured releasably to the outer wall of the drinking straw. This can be effected by a shrink foil for example, in particular by a shrink sleeve or a shrink cap, as closure element. The closure element can moreover be fixed with an additional adhesive tape on the outer wall of the drinking straw.

In order to protect the membrane, the membrane-side end of the wall can be closed by a barrier layer. The barrier layer should be airtight, such that air cannot inadvertently be sucked in through the drinking straw. This could lead to the membrane accidentally being torn by an intake of air. In this case, there is a danger of the patient inhaling the finely powdered active substance. At the same time, the barrier layer should be water-soluble, such that it dissolves upon contact with a drink. In this way, the full functionality of the drinking straw can be ensured after the drinking straw has been standing for a short time in a drink. After the barrier layer has dissolved, the drink can be sipped through the drinking straw, thus also allowing the active substance to be taken. The barrier layer can be made in particular of a taste-neutral polymer, for example gelatin.

The wall of the drinking straw according to the invention can have a reinforcement at the first and/or second end, as is already known from WO 2006/079648 A1, for example.

A drinking straw within the meaning of this invention is any drinking straw with which a liquid can be sipped through the mouth of a human being.

The drinking straw according to the invention can preferably be made of a thermoplastic material or thermosetting plastic. If the content of the drinking straw is intended to be visible, the wall of the drinking straw can be made of a transparent material, in particular of a colourless transparent material. In this way, it can be immediately seen whether the active substance is still present in the drinking straw or has already been consumed.

Further advantages and features of the invention will be gathered from the features additionally indicated in the claims and from the illustrative embodiments set out below.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1 shows a longitudinal section through a first embodiment of the drinking straw according to the invention, before the active substance starts to be drawn up;

FIG. 2 shows a longitudinal section through the drinking straw from FIG. 1, after the active substance has been drawn up;

FIG. 3 shows a transverse section through the drinking straw from FIG. 1 along the line A-A;

FIG. 4 shows a longitudinal section through a second embodiment of the drinking straw according to the invention, before the active substance starts to be drawn up;

FIG. 5 shows a longitudinal section through the drinking straw from FIG. 4, after the active substance starts to be drawn up;

FIG. 6 shows a plan view of the intact membrane of the drinking straw from FIG. 4;

FIG. 7 shows a plan view of the torn open membrane of the drinking straw from FIG. 5;

FIG. 8 shows a longitudinal section through a third embodiment of the drinking straw according to the invention, before the active substance starts to be drawn up;

FIG. 9 shows a longitudinal section through the drinking straw from FIG. 8, after the active substance starts to be drawn up;

FIG. 10 shows a plan view of the intact membrane of the drinking straw from FIG. 8;

FIG. 11 shows a plan view of the torn open membrane of the drinking straw from FIG. 9;

FIG. 12 shows a perspective view of the upper end of the drinking straw, which is closed with a sealed-on foil; and

FIG. 13 shows a perspective view of the upper end of the drinking straw, which is closed with a heat-shrink cover.

### DETAILED DESCRIPTION OF PREFERRED EMBODIMENTS

FIGS. 1 to 3 show a first embodiment of the drinking straw 10 according to the invention for administering an active substance 12. The drinking straw 10 has a wall 20 with a first, lower end 22 and a second, upper end 24. The lower end 22 can be placed into a vessel containing a drinkable liquid. The upper end 24 is placed into the mouth, such that the liquid can be drawn up through the drinking straw 10.

Before use, the upper end 24 of the wall 20 is closed by a closure element in the form of a cap 30. The cap 30 sits with frictional engagement on the upper end 24 of the wall 20. In this way, the active substance located in the interior 32 of the wall 20 cannot trickle out from the top of the drinking straw 10.

A membrane 40 is present in the region of the lower end 22 of the wall 20. The membrane 40 is sufficiently tight to ensure that even finely powdered active substances 12 cannot trickle through the membrane 40. At the same time, the membrane 40 is so smooth that the active substances 12 cannot adhere to the membrane 40. The membrane 40 thus allows finely powdered formulations containing active substance to be administered through the drinking straw 10 according to the invention. The active substance formulations can contain an individual active substance or else a mixture of different active substances. In both cases, the formulations can also contain additional fillers besides the active substances.

In the present example, the membrane 40 is secured to the inner wall 42 of the drinking straw 10. The membrane 40 is tensioned here via a clamping element in the form of a tubular fitting 44. The tubular fitting 44 has an external diameter 46 that is slightly smaller than the internal diameter 48 of the wall 20 of the drinking straw 10. A continuous slit 50 is formed in the longitudinal direction 52 in the tubular fitting 44. As a result of this slit 50, the tubular fitting 44 can expand farther out such that it bears sealingly on the inner face 42 of the wall 20. The membrane 40 located between the outer face of the tubular fitting 44 and the inner face 42 of the wall 20 is thus fixed at the desired position.

The active substance 12 to be administered can lie on the membrane 40. The active substance 12 is then located in the interior of the drinking straw 10 between the membrane 40 and the cap 30 (see FIG. 1). For transporting and storing the drinking straw 10, the latter can additionally be packed in external packaging (not shown here), for example in a film bag.

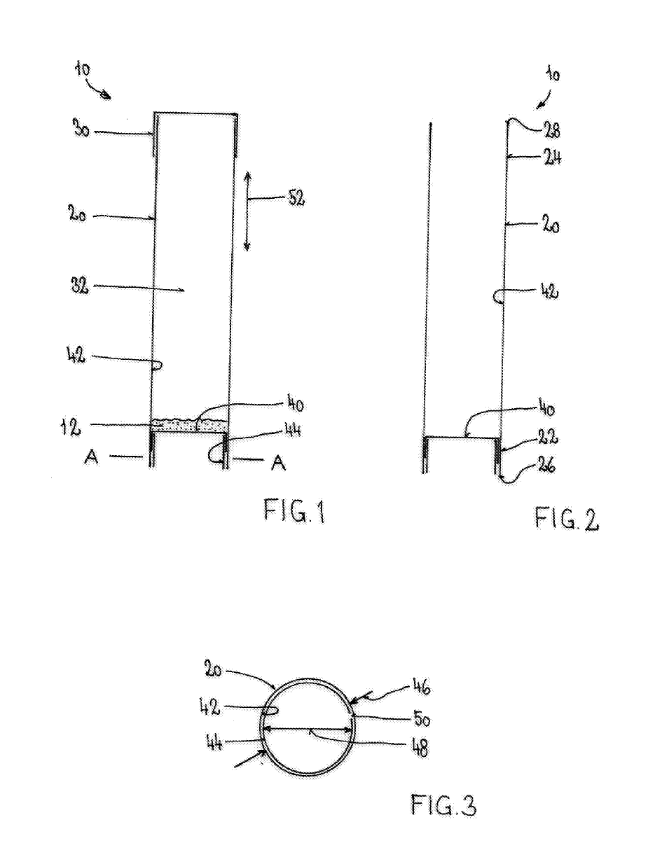

In the illustrative embodiment according to FIGS. 1 to 3, the membrane 40 is permeable to liquid. The liquid to be drunk can thus be sucked through the membrane 40. The active substance 12 to be administered can in this case be dissolved or suspended in the liquid and in this way enter the patient's mouth. The membrane 40 remains undamaged and is unchanged after completion of the drinking process (see FIG. 2).

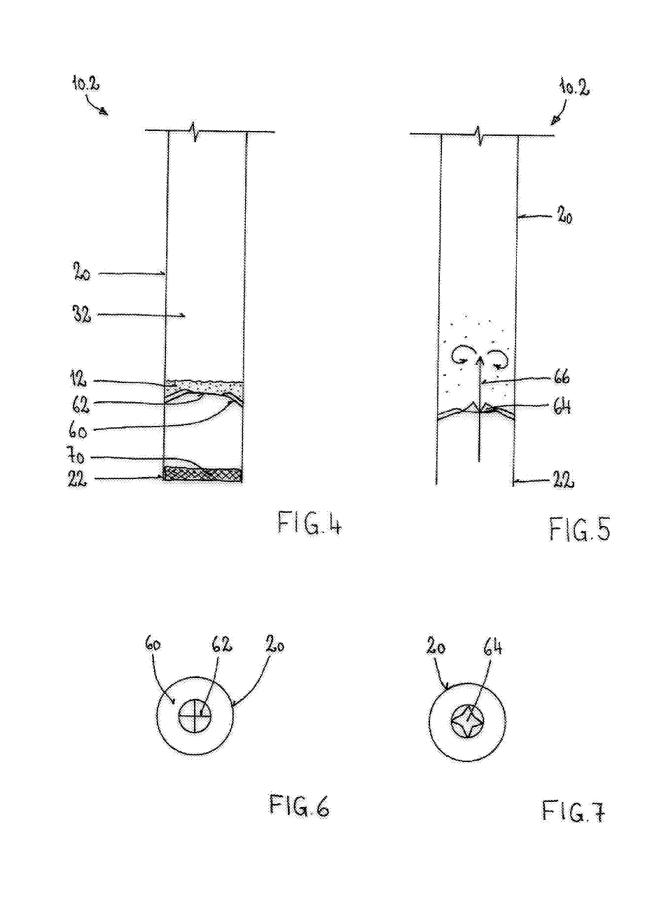

FIGS. 4 to 7 show a second embodiment of the drinking straw 10.2 according to the invention. The membrane 60 is in this case welded to the inner wall 42 of the drinking straw 10.2. The membrane 60 has a predetermined breaking point in the form of a material thinning 62. The material thinning 62 can be produced by means of a laser, for example. Individual layers of the membrane 60 can be removed by a laser in such a way that that, although still being able to serve as a seal for the active substance 12 to be administered, the membrane nonetheless tears when subjected to a compressive load or suction load.

In the present example, the material thinning 62 is provided in the central region of the membrane 60. The membrane 60 thus tears at the centre, while it remains firmly secured at its edge to the inner wall 42 of the drinking straw 10.2. This can ensure that the membrane 60 does not accidentally tear in such a way that parts of the membrane 60 are also sucked in. In the present example, the material thinning 62 is cross-shaped (see FIG. 6), such that a plane opening 64 forms (see FIG. 7) as soon as the membrane 60 starts to tear. The liquid can be drawn up (arrow 66) through this opening 64. The active substance 12 to be administered is entrained with the liquid and can likewise be drawn up and swallowed.

Like the membrane 40, the membrane 60 is also sufficiently tight to ensure that even finely powdered active substances 12 cannot trickle through the membrane 60. At the same time, the membrane 60 is so smooth that the active substances 12 cannot adhere to the membrane 60.

To prevent the membrane 60 from starting to tear too early, a barrier layer 70 is provided at the membrane-side lower end 22 of the wall 20 in the present illustrative embodiment. The barrier layer 70 closes off the interior 32 of the drinking straw 10 in an airtight manner at the bottom. In this way, it is possible to prevent the membrane 60 from already tearing during the aspiration of air, in which case the active substance 12 could be accidentally inhaled. The barrier layer 70 dissolves after standing for a short time in a drink. The drink can then be drawn up through the drinking straw 10. Only then does the membrane 60 tear, and the active substance 12 can be drawn up and swallowed.

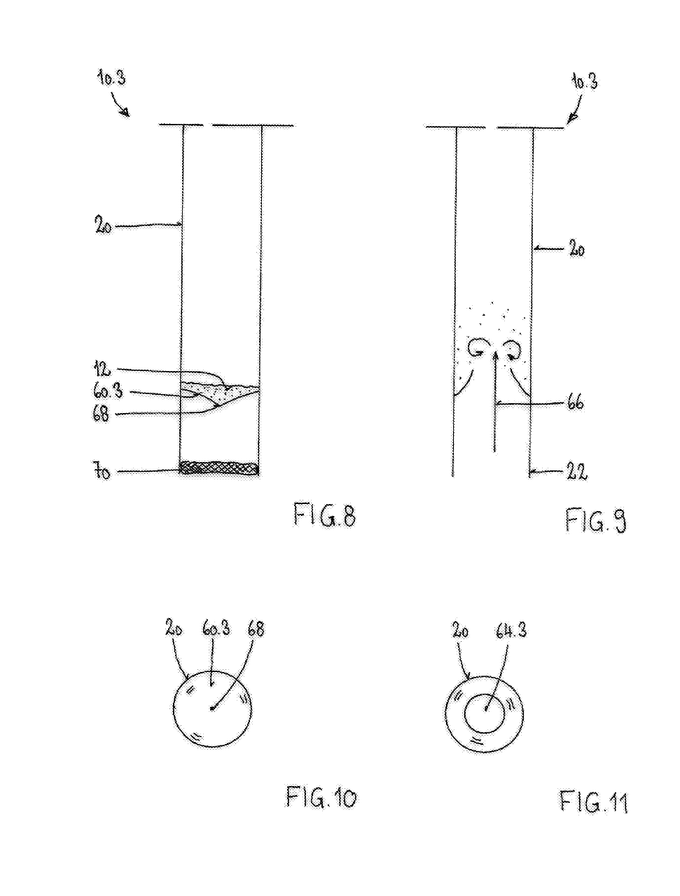

FIGS. 8 to 11 show a third embodiment of the drinking straw 10.3 according to the invention. In this case too, the membrane 60.3 is secured to the inner wall 42 of the drinking straw 10.3. The membrane 60.3 has a predetermined breaking point in the form of a laser perforation 68 (see FIG. 10). When subjected to a compressive load or suction load, the laser perforation 68 tears, and a circular opening 64.3 (see FIG. 11) forms in the central region of the membrane 60.3. The liquid can be drawn up (arrow 66) through this opening 64.3. The active substance 12 to be administered is entrained with the liquid and can likewise be drawn up and swallowed. In this embodiment too, the barrier layer 70 is provided at the outset at the lower end 22 of the wall 20 (see FIG. 8).

The barrier layer 70 could also be provided in the first illustrative embodiment according to FIGS. 1 to 4.

In contrast to the illustrative embodiments shown here, the membrane 40, 60, 60.3 could also be secured to the front face 26 of the lower end 22 of the wall 20.

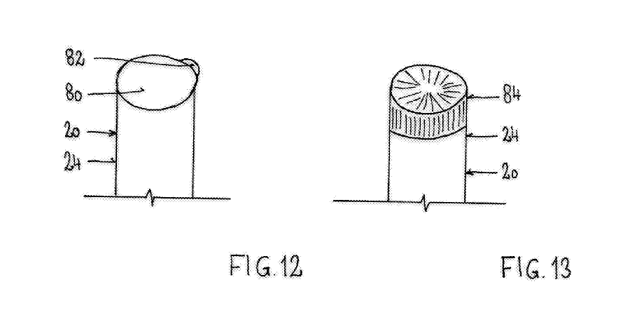

FIGS. 12 and 13 show two alternative closure elements for the upper end 24 of the wall 20. According to FIG. 12, the closure element is configured as a sealing foil 80. The sealing foil 80 is secured releasably to the front face 28 of the upper end 24 of the wall 20. The sealing foil 80 has a tab 82 for this purpose. By means of the tab 82, the sealing foil 80 can be pulled away from the front face 28 in order to allow the active substance 12 to be drawn up.

According to FIG. 13, the closure element 84 is configured as a shrink cap 84. The shrink cap 84 can be pulled away from the wall 20 in order to expose the upper end 22 of the wall 20. As an alternative to this, the shrink cap 84 could also have one or more perforations that may make it easier to tear open and thus remove the shrink cap 84. Instead of the shrink cap 84, a shrink sleeve could also be used for example.

Although only a few embodiments of the present invention have been shown and described, it is to be understood that many changes and modifications may be made thereunto without departing from the spirit and scope of the invention.

What is claimed is:

1. A drinking straw for administering an active substance,
having a wall with a first end and with a second end,
having a closure element with which one of the two ends of the wall can be releasably closed,
wherein
a membrane is present in the region of the other end of the wall,
the active substance lies on the membrane.

2. The drinking straw according to claim 1, wherein the membrane is permeable to liquid.

3. The drinking straw according to claim 1, wherein an opening through which liquid is sucked can be formed in the region of the membrane.

4. The drinking straw according to claim 3, wherein the membrane has at least one predetermined breaking point, which is configured in particular as a material thinning.

5. The drinking straw according to claim 1, wherein the membrane is secured to the inner wall of the drinking straw.

6. The drinking straw according to claim 5, wherein the membrane is secured to the inner wall of the drinking straw by means of a clamping element.

7. The drinking straw according to claim 6, wherein the clamping element has a tubular fitting, the external diameter of which is slightly smaller than the internal diameter of the wall of the drinking straw, and wherein the tubular fitting has a continuous slit in the longitudinal direction.

8. The drinking straw according to claim 1, wherein the closure element is configured as a foil.

9. The drinking straw according to claim 8, wherein the closure element is releasably secured, in particular bonded or welded, to the front face of the wall.

10. The drinking straw according to claim 8, wherein the closure element is releasably secured to the outer wall of the drinking straw.

11. The drinking straw according to claim 10, wherein the closure element is configured as a shrink foil, in particular as a shrink cap or as a shrink sleeve.

12. The drinking straw according to claim 1, wherein the membrane-side end of the wall is closed in an airtight manner by a water-soluble barrier layer.

13. The drinking straw according to claim 12, wherein the water-soluble barrier layer is made of a polymer, in particular of gelatin.
